(19) United States
(12) Patent Application Publication
(10) Pub. No.: US 2006/0100588 A1
(43) Pub. Date: May 11, 2006

(54) Needle handling device

(76) Inventors: Lennart Brunnberg, Tyreso (SE); Victor Kronestedt, Stockholm (SE); Jochen Ratjen, Nacka (SE)

Correspondence Address: YOUNG & THOMPSON, 745 SOUTH 23RD STREET, 2ND FLOOR, ARLINGTON, VA, 22202, US

(21) Appl. No.: 10/896,983

(22) Filed: Jul. 23, 2004

(51) Int. Cl. A61M 5/32

(52) U.S. Cl. 604/192; 128/919

(57) ABSTRACT

A device for handling injection needles, especially in relation to injectors, includes grip elements for gripping, removing and retaining a sheath surrounding a needle attached to an injector device, guide elements for guiding the sheath back onto the needle after completed injection, elements for removing the needle from the injector device and activation elements capable of acting on the grip elements for removing the needle from the device.

### TECHNICAL AREA

The present invention relates to a device for handling injection needles and in particular attaching and detaching of such needles in a safe way.

### BACKGROUND OF THE INVENTION

A secure handling of needles has become a very important topic because of the health hazards with used needles, which may transmit infections from patient to hospital staff. Also for devices for self administration, such as pen injectors and the like handled by the patients themselves, it is important that no one can become injured by accidental needle sticks.

For safety and convenience reasons it is also desirable to provide a safe handling of the needle before use, e.g. when attaching the needle to a delivery device.

A very large number of devices have been developed in order to obtain safe handling of such needles such as holders adapted to fit and grip a needle before and/or after its use, means for discarding used needles, needle shields that are attached to the delivery device and which surrounds the needle after injection in a shielding and possibly locking manner.

One area where needles have to be handled and replaced is multi-dose injectors for delivering several doses before the injector is discarded. After each injection the needle is removed and before each injection a new needle is attached.

One example of a device for handling needle attachment/detachment is disclosed in U.S. Pat. No. 5,968,021. The device comprises a needle unit comprising a needle arranged in a sleeve. The unit is arranged in a needle cover. In order to attach the needle on a syringe or a cartridge, the housing is pushed over a connection piece whereby two inwardly directed protrusions fit into a groove and holds the sleeve. The needle shield may then be pushed off. After injection the needle shield is pushed onto the sleeve but in a different rotational position in relation to this whereby protrusions on the outside of the sleeve are pressed against the inside of the needle cover. This leads to that the inwardly directed protrusions are pushed out of the groove due to elastic deformation and the needle unit can be removed from the syringe or cartridge.

However many needle units have an “inner” needle sheath which fits rather tightly around the needle or a needle hub and is often made of a rather thin and resilient material. The needle and the inner sheath is then positioned in an outer cover of a more rigid material, which cover together with a tear-off sealing foil keeps the needle sterile until use.

The outer cover could be used in the same manner as the above described needle shield for attaching the needle unit to a delivery device. However, when the outer cover is removed the patient or the operator has to remove the inner sheath by hand, whereby there is a risk for accidental needle sticks. Also many users feel a discomfort in having their hands in the vicinity of the needle.

Further, many of the outer covers on the market are not intended, or not suitable, for removing the needle and/or for covering the needle after use. For an injector where the needle has to be replaced for a subsequent injection this implies that either the user has to remove the unprotected needle by hand or to try to push the inner needle sheath onto the needle before removal, in the cases a needle shield of an injector does not allow for using the outer cap of the needle, or to push on the outer cap. When attempting to push on either of the caps there is an obvious risk of having the needle penetrating the cap by its sharp tip hence causing unintentional needle sticks.

Also if now the needle has become bent during handling and use, it may penetrate the sheath when the sheath is returned onto the needle after use. It may also penetrate the outer cap if that is used for removing the needle. Hence a risk for accidental needle sticks is obvious.

There is thus a need for a handling device which is capable of attaching the needle in a safe way and after use remove and discard the needle in a convenient and safe way, with some kind of protection surrounding the needle, protecting persons handling the discarded needle.

### BRIEF DESCRIPTION OF THE INVENTION

The aim of the present invention is to solve the above mentioned problems in connection with needle handling.

This aim is solved with the present invention characterised in the features of claim 1. Preferable embodiments of the invention are found in the depending claims.

According to a main aspect of the invention it is characterised by a device for handling injection needles, especially in relation to injectors, comprising grip means for gripping, removing and retaining a sheath surrounding a needle attached to an injector device, guide means for guiding the sheath back onto the needle after completed injection, means for removing said needle from said injector device and activation means capable of acting on the grip means for removing said needle from said device.

According to another aspect of the invention said grip means comprises radially displaceable tongues and means capable of displacing the tongues radially inwards when the device is pushed onto the needle/sheath.

According to a further aspect of the invention said displacing means comprises a housing surrounding said tongues and being slidable in relation to the longitudinal direction of the tongues, and in inclined surfaces on the inside of the sleeve and outside of the tongues, wherein when the tongues slide in relation to the housing they are forced radially inwards in order to grip the sheath.

Preferably the device comprises lock means capable of locking the tongues in the grip position in relation to the housing.

According to yet an aspect of the invention the guide means comprises inclined surfaces sloping inwards towards a centre line between the tongues.

According to another aspect of the invention said needle removing means comprises contact means capable of co acting with corresponding contact means arranged on an outer surface of an attachment part of the needle unit arranged on an inner surface of the housing, wherein, when in contact, the contact means of the housing is capable of removing said needle unit from the injector.

According to yet another aspect of the invention it comprises actuating means capable of releasing said lock means and acting on the grip means for releasing and discarding said needle/sheath from the device.

The advantages with the present invention is that the inner sheath, very often present with needle units intended for in particular injectors with a certain degree of automatic functions, can be removed without the hands of the user being in the vicinity of the needle. Further, the device can retain the sheath and is capable of reintroducing the sheath onto the needle before the needle unit is removed from the injector.

A further advantageous feature is that the present device preferably is adapted to be able to remove the needle unit from the cartridge and the like from the injector after the sheath is inserted onto the needle. The needle unit can then be discarded from the device where the needle is protected by the surrounding sheath, and thrown into a suitable container, depending on local legislation preferably a container for health hazardous items.

The device is then ready to be used again. Apart from being able to remove the inner sheath and replacing it after injection, and also to remove the needle unit, the device of the present invention is intended to be used a large number of times, as compared to the conventional devices which are used only once for removing a needle unit from an injector after completed injection. The device is thus safe as regards handling of an injection needle as well as being environmentally friendly regarding the multiple uses.

These and other aspects of and advantages with the present invention will become apparent from the detailed description of the invention and from the appended drawings.

### DETAILED DESCRIPTION OF THE DRAWINGS

In the following detailed description of the invention reference will be made to the accompanying drawings, of which

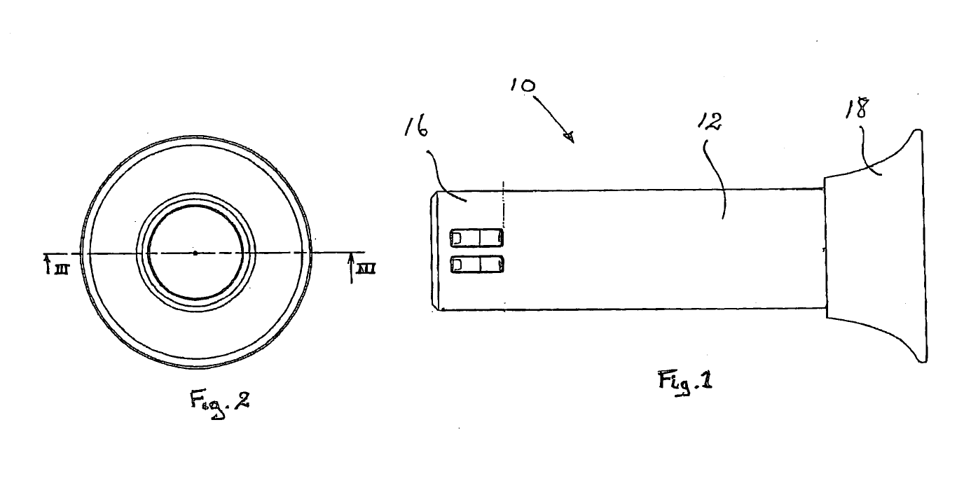

FIG. 1 is a side view of the device according to the invention,

FIG. 2 is an end view of the device according to claim 1,

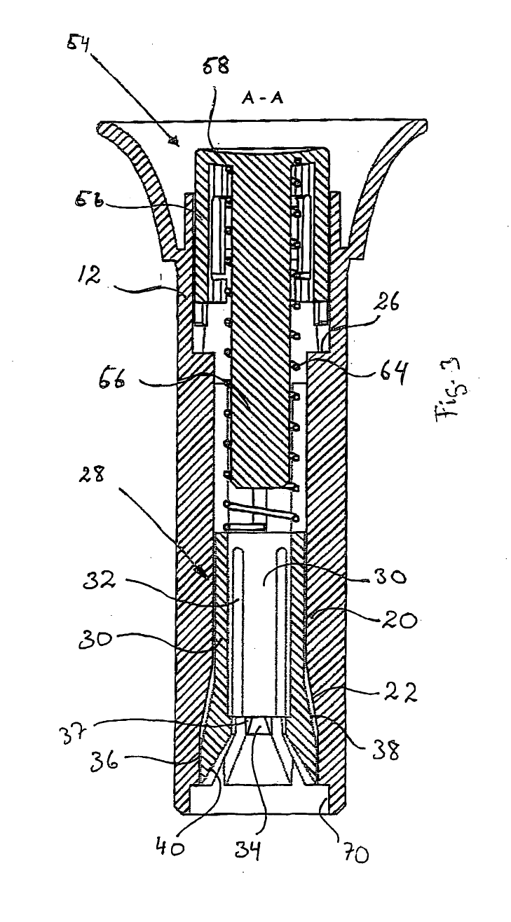

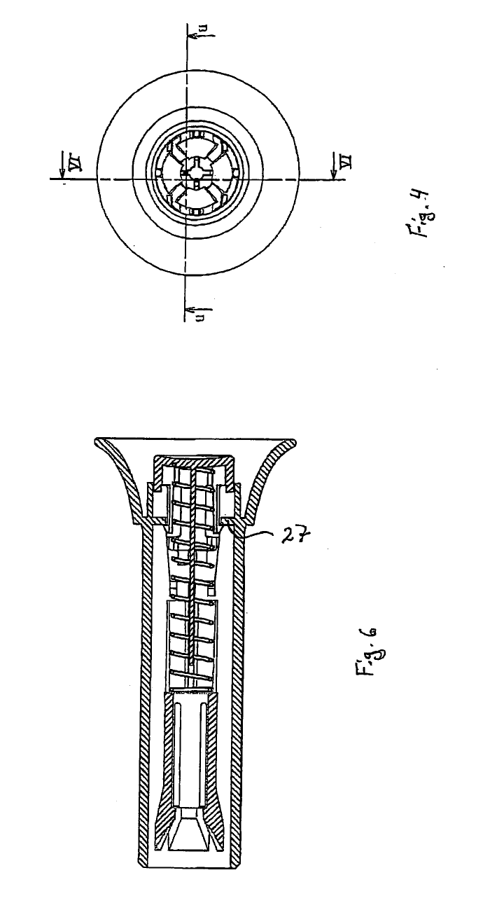

FIG. 3 is a cross-sectional longitudinal view of the device in a first state taken along the line III-III of FIG. 2,

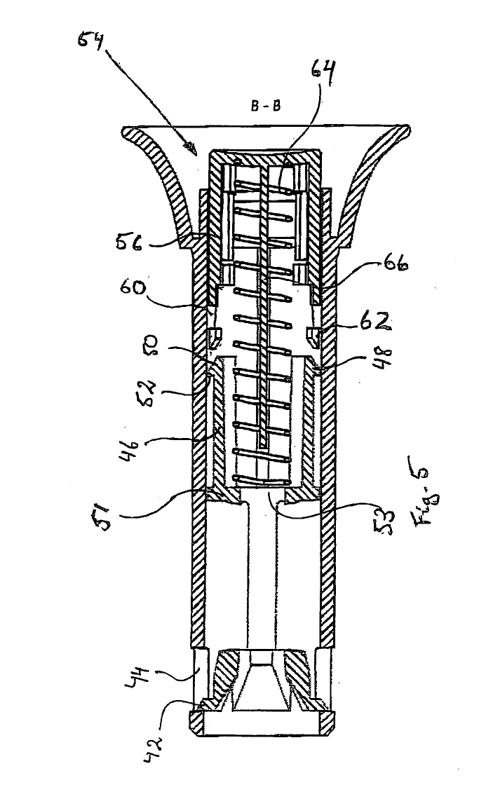

FIG. 4 is an end view of the opposite end in relation to FIG. 2,

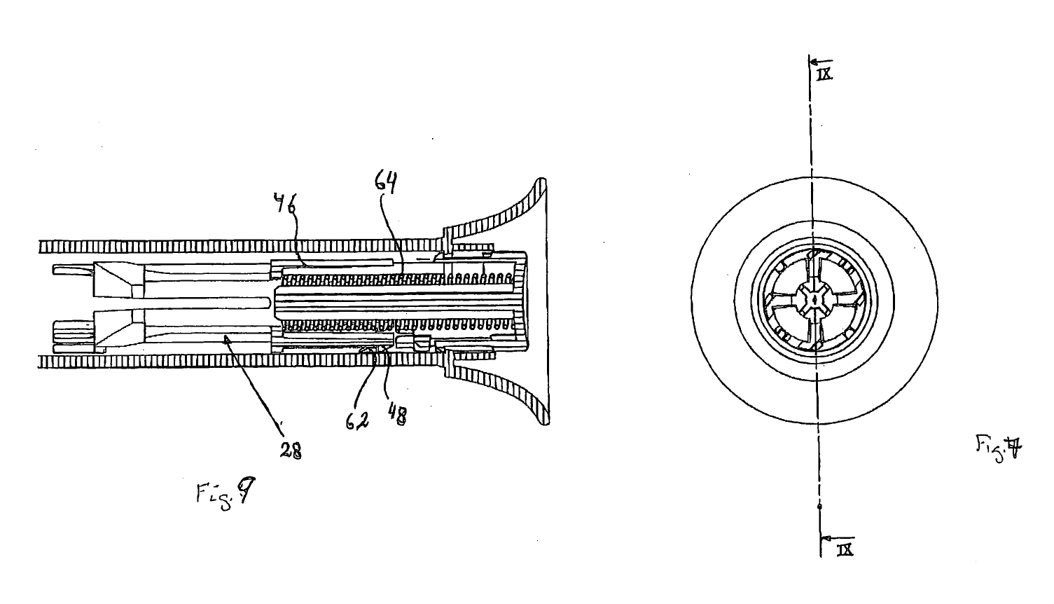

FIG. 5 is a cross-sectional longitudinal view of the device in a first state taken along the line V-V of FIG. 4,

FIG. 6 is a cross-sectional longitudinal view of the device in a first state taken along the line VI-VI of FIG. 4,

FIG. 7 is an end view corresponding to FIG. 4,

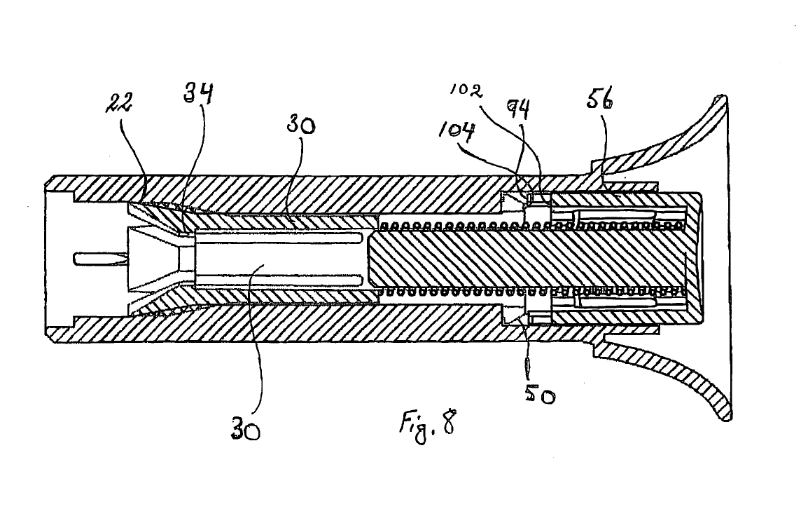

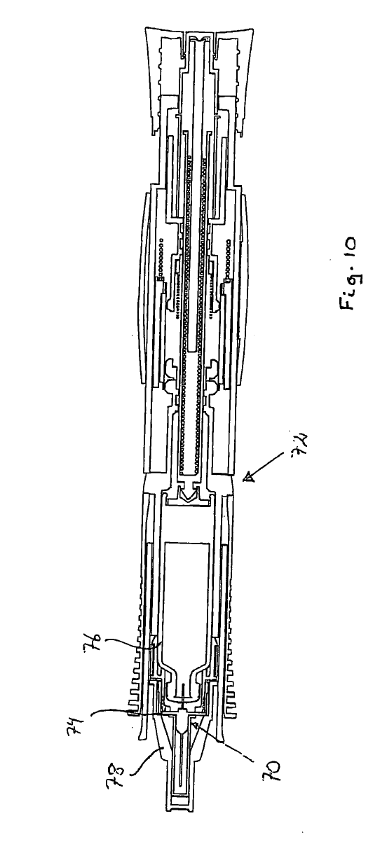

FIG. 8 is a cross-sectional longitudinal view of the device in a second state taken along the line VIII-VIII of FIG. 7,

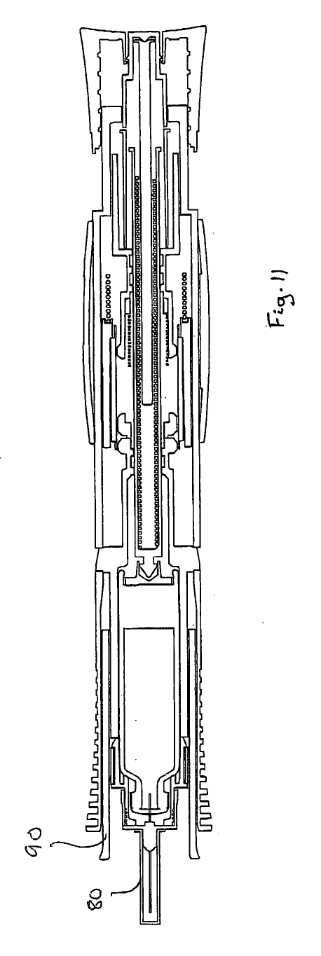

FIG. 9 is a cross-sectional longitudinal view of the device in a second state taken along the line IX-IX of FIG. 7,

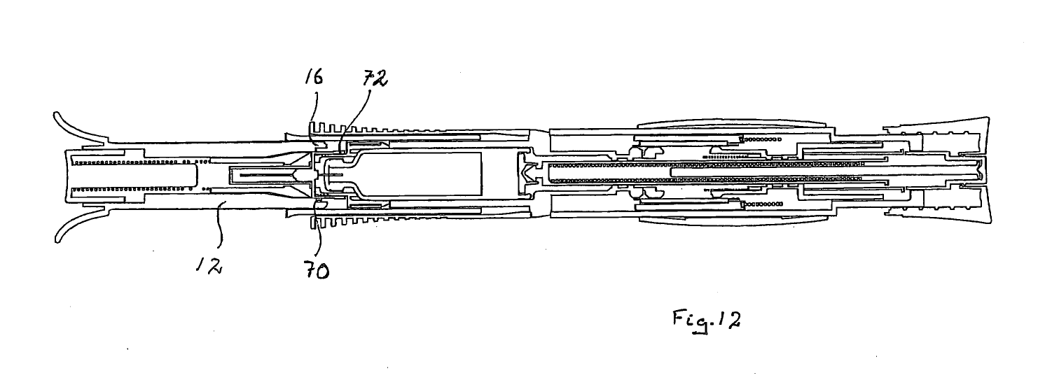

FIG. 10-16 are cross-sectional views of the sequence of operation of the device according to the present invention, and

FIG. 17 is a variant of the device according to FIGS. 1-16.

### DETAILED DESCRIPTION OF THE INVENTION

FIGS. 1 and 3 show side views of the device 10. It comprises an outer substantially cylindrical housing 12 acting as a grip, with a front end 16, to the left in the figures, having a smooth surface, and a rear end 18, which is widened in order to provide a grip. The function of the rear and front ends will be explained below. The inner surface of the housing 12 is arranged with four longitudinally extending ribs 20, where two are shown in FIG. 3, arranged equally spaced around the inner circumference. The front ends of the ribs 20 are arranged with sloping surfaces 22. The inner ends each of the ribs are further arranged with a ledge 26. Protrusions 27 are arranged inside the housing at the rear part, FIG. 6.

Inside the housing a grip means 28 is slidably arranged. It comprises a front part of a generally cylindrical shape divided in four arms 30 by four grooves 32. The front end of each arm 30 is arranged with inwardly directed protrusions 34, outwardly directed protrusion 36 and a sloping transition 38, and the front end of each arm 30 has somewhat inwards sloping surfaces 40. The inwardly directed protrusions 34 are preferably arranged with sharp, triangularly shaped edges 37. Some of arms 30 are also provided with outwardly directed ledges 42, FIG. 5, that fit into cut-outs 44, limiting the axial movement of the grip means in relation to the housing.

The rear part of the grip means 28 is arranged with four rearwardly extending arms 46, FIG. 5, where each arm is arranged with outwardly directed protrusions or hooks 48 having a sloping surface 50 in the rearward direction and a ledge 52 in the forward direction. Between the front arms 30 and the rearwardly extending arms 46 a dividing wall 51 is arranged, having a central opening 53, FIG. 5.

At the rear end of the housing an actuating means 54, in the embodiment a push button, is arranged comprising an outer cylindrical part 56 with an end wall 58 somewhat protruding out of the rear part of the housing. The cylindrical part is arranged with grooves, the position of which corresponds to the position of the protrusions 27 of the inner surface of the housing. A number of tongues 60, FIG. 5, extend into the corresponding grooves, where the end of each tongue is arranged with inwardly extending ledges 62. The number and positions of the tongues 60 correspond to the number and positions of the arms 46 of the grip means 28. A spring 64 is arranged between the inner surface of the end wall 58 and the dividing wall 51. A guide means 66 is attached to the end wall 58 and runs inside the spring 64.

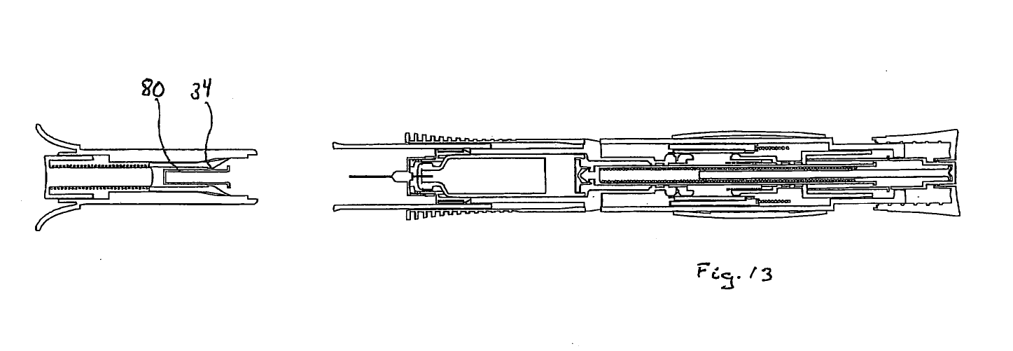

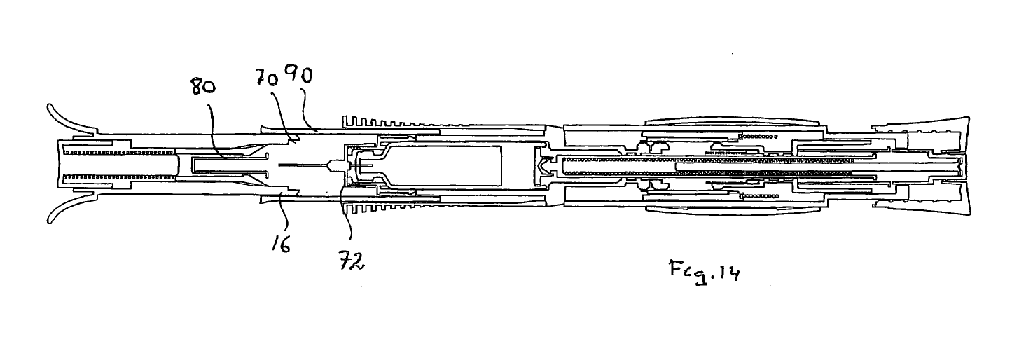

The device is intended to function as follows. A needle unit 70 is mounted onto a delivery device such as an auto injector 72, FIG. 10, e.g. by screwing it onto the threaded front part 74 of the medicament cartridge 76, by using the outer cover 78 that the needle unit is delivered in, and which outer cover keeps the needle sterile together with a tear-away foil. When the needle unit is attached the outer cover is removed, FIG. 11. The inner sheath 80 now has to be removed from the needle. The device according to the invention is now used by pushing the front part of the housing onto the needle and sheath, FIG. 12, where the grip means 28 is in its forward position, FIGS. 2-6. The sloping surfaces 40 of the front ends of the arms 30 aid in guiding the sheath inside the grip means. The front end 16 of the housing 12 is preferably arranged with a recess 70 corresponding to the attachment part of the needle unit and when the device is pushed fully onto the needle/sheath the attachment part or needle hub 72, FIG. 12, is arranged in the recess. During this operation the needle hub 72 of the needle unit pushes against the front ends of the grip means, thereby pushing it into the housing against the force of the spring 64. This movement causes the sloping surfaces 22 of the ribs 20 to act on the sloping surfaces 38 of the arms 30, thereby pressing them radially inwards, FIG. 8. This in turn causes the inwardly protrusions 34 of the arms to be pressed against the sheath 80 and to provide a mechanical grip of the sheath, FIG. 13, where the sharp edges 37 contribute to a secure gripping action. The rearward movement of the grip means 28 also causes the outwardly directed protrusions 48 of the arms 46 to move past the ledges 62 of the actuating means, whereby the arms 46 flex inwardly and the protrusions 48 lock with the ledges 62, FIG. 9. The grip means 28 is now locked axially in relation to the housing 12 and the sheath is locked between the arms of the grip means. The device 10 is now removed, bringing the sheath with it off the needle, and the injector is ready for an injection, FIG. 13.

If an injector is used having a needle shield 90, FIG. 11, axially movable in relation to the injector and protruding somewhat out of and surrounding the attachment end of the cartridge, the front end 16 of the device is adapted such that the smooth outer surface of the front end has a diameter somewhat smaller than the inner diameter of the needle shield 90 of the injector. This further aids in guiding the device onto the needle/sheath so that the proper position is obtained between the sheath and the arms of the grip means. Should the needle however penetrate the sheath, there is no risk for injury for the patient or person handling the device, since the needle is well confined within the housing of the device.

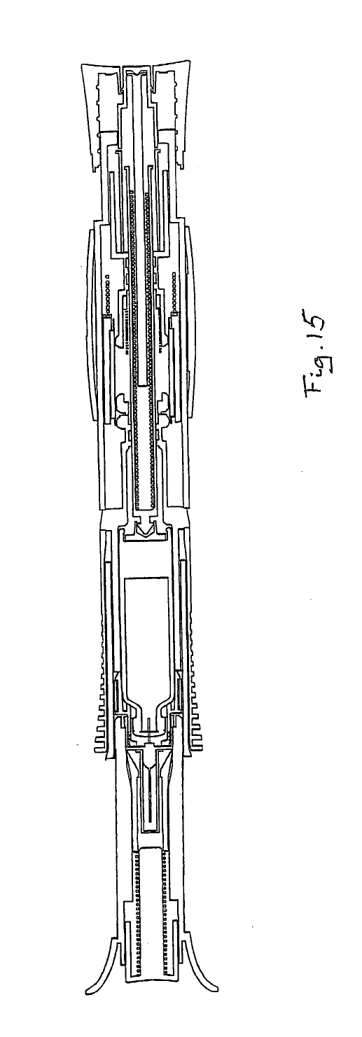

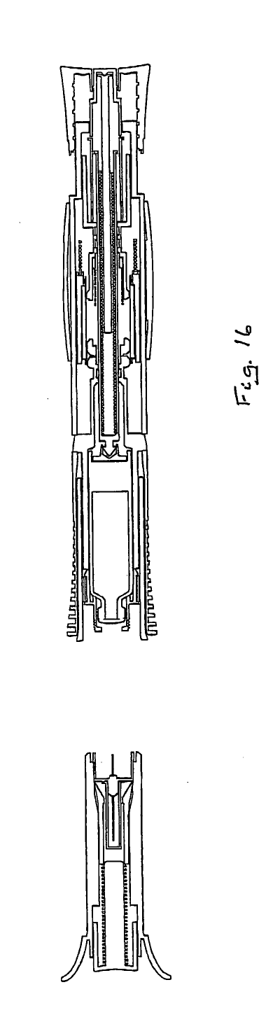

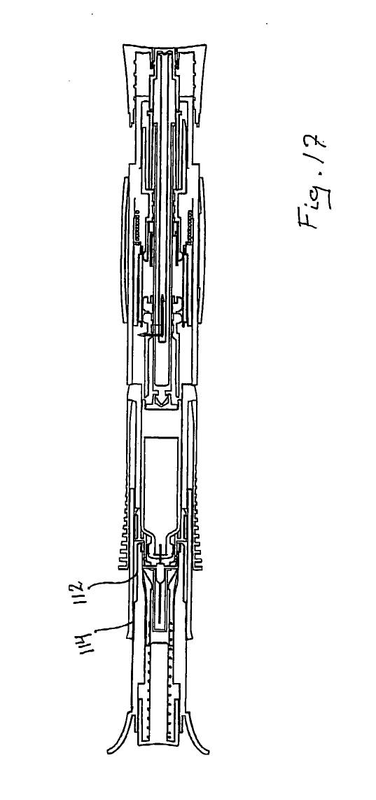

When the injection is completed and the needle unit is to be removed from the injector, the front end 16 of the device 10 is again directed onto the needle whereby the sheath is pushed onto the needle, FIG. 14. Again, if a needle shield 90 is provided on the injector, this is in its extended position after injection, thereby surrounding the needle, the front part 16 of the device is guided by the needle shield, and when the device pushes the needle shield into the injector, the proper position between the grip means/sheath and the needle is ensured. The device is pushed until the attachment part of the needle unit is positioned inside the recess 70 of the front part, FIG. 15. Preferably the outer sure of the needle hub 72 of the needle unit is arranged with protrusions, normally longitudinally extending ribs, intended to be used as grip means for manually screwing on and off the needle unit from the cartridge, and the inner surface of the recess 70 is arranged with corresponding mating recesses in order to obtain a rotational lock between the attachment part and the housing. The user can now unscrew the needle unit from the cartridge by turning the device and pulling, thereby removing the needle unit from the injector, FIG. 16. There could of course also be a frictional lock between the needle unit and the device.

In order to discard the needle unit from the device, the user presses the end wall 58 of the actuator means 54 inside the housing 12. The sloping surfaces 50 of the rearwardly ding arms 46 will then come in contact with lower end 94 of the cylindrical part 56, FIG. 8, forcing the arms 46 to move radially inwards until the protrusions 48 loose contact with the ledges 62 and the end surfaces of the grooves of the cylindrical part abuts the protrusions 27 of the housing. The pushing force from the user and freeing of the arms in combination with the force of the spring 64 causes the grip means 28 to be pushed forward rather quickly, whereby the needle unit is be “ejected” out of the device and into a proper receptacle for storage. If the force of the spring for some reason is insufficient, further pressing of the actuator means will cause an inner edge 102, FIG. 8, to act on an upper surface 104 of the rearwardly extending arms 50, thus ensuring that the needle is pushed out of the device.

A new needle unit can now be attached to the injector in the same manner as described above. It is thus possible to use the device several times to remove a needle unit onto one or several injection devices.

It is to be understood that the embodiments described above and shown in the drawings are to be regarded as non-limiting examples of the present invention and that it may be modified within the scope of the claims.

A variant of the described device is shown in FIG. 17, where the housing is adapted to remove a needle unit from an injection device where the needle shield is locked in the extended position, which is often the case after an injection in order to protect the user or other persons from unintentional needle sticks. As can be seen from FIG. 17 the front end of the housing is arranged with a section 112 having a first diameter, which is somewhat smaller than the inner diameter of the needle shield, followed by a section 114 having a diameter more or less corresponding to the inner diameter of the needle shield.

Even though the needle unit has been described with threads for engaging with the cartridge, it could be provided with other attachment means, wherein the device according to the present invention could be provided with corresponding contact means capable of removing the needle unit. It is also conceivable that the grip means is arranged and designed such that any gripping action of the sheath is obtained. Further the actuating means could also be a sliding button, lever or any other mechanical activating member.

1. Device for handling injection needles, especially in relation to injectors, comprising grip means for gripping, removing and retaining a sheath surrounding a needle attached to an injector device, guide means for guiding the sheath back onto the needle after completed injection, means for removing said needle from said injector device and activation means capable of acting on the grip means for removing said needle from said device.

2. Device according to claim 1, wherein said grip means comprises radially displaceable tongues and means capable of displacing the tongues radially inwards when the device is pushed onto the needle/sheath.

3. Device according to claim 2, wherein said displacing means comprises a housing surrounding said tongues and being slidable in relation to the longitudinal direction of the tongues, and in inclined surfaces on the inside of the sleeve and outside of the tongues, wherein when the tongues slide in relation to the housing they are forced radially inwards in order to grip the sheath.

4. Device according to claim 3, further comprising lock means capable of locking the tongues in the grip position in relation to the housing.

5. Device according to claim 2, wherein the guide means comprises inclined surfaces sloping inwards towards a centre line between the tongues.

6. Device according to claim 2, wherein said needle removing means comprises contact means capable of co acting with corresponding contact means arranged on an outer surface of an attachment part of the needle unit arranged on an inner surface of the housing, wherein, when in contact, the contact means of the housing is capable of retaining contact between the needle unit and the device, thereby enabling removal of said needle unit from the injector.

7. Device according to claim 4, wherein it comprises actuating means capable of releasing said lock means and acting on the grip means for releasing and discarding said needle/sheath from the device.

8. Device according to claim 7, wherein said actuating means is a push button or sliding button.
